(19) United States
(12) Patent Application Publication
(10) Pub. No.: US 2024/0285729 A1
(43) Pub. Date: Aug. 29, 2024

(54) METHODS AND KITS FOR INDUCING SATIETY AND TREATING METABOLIC DISORDERS

(71) Applicant: Gila Therapeutics, Inc., Minneapolis, MN (US)

(72) Inventor: Thomas VASICEK, Minneapolis, MN (US)

(21) Appl. No.: 18/572,395

(22) PCT Filed: Jun. 24, 2022
(86) PCT No.: PCT/US2022/034836

§ 371 Date: Dec. 20, 2023

Related U.S. Application Data
(60) Provisional application No. 63/214,382, filed on Jun. 24, 2021.

(51) Int. Cl. A61K 38/22; A61K 9/00; A61K 38/26

(52) U.S. Cl. CPC A61K 38/22; A61K 9/006; A61K 38/26

(57) ABSTRACT

The invention provides methods and kits for inducing satiety and treatment of metabolic disorders, diabetes, obesity, and obesity-related conditions. The methods and kits herein described include administration of a metabolic hormone and a GLP-1 receptor agonist for inducing satiety or treatment of metabolic syndrome, diabetes, obesity, and obesity-related disorders.

### SEQUENCE LISTING

The instant application contains a Sequence Listing which has been submitted electronically in ASCII format and is hereby incorporated by reference in its entirety. The ASCII copy, created on Jun. 22, 2022, is named 51547-007WO2_Sequence_Listing_6_22_22_ST25 and is 24,476 bytes in size.

### BACKGROUND OF THE INVENTION

The prevalence of obesity continues to increase worldwide. However, there is still a lack of effective, long-term, non-invasive treatments for obesity and other metabolism related disorders. Induction of satiety and treatment of metabolic syndrome, diabetes, obesity, and obesity-related disorders with existing therapeutics often does not produce suitable therapeutic effects. Accordingly, new treatments are needed.

### SUMMARY OF THE INVENTION

The invention provides methods and kits for inducing satiety and treatment of metabolic disorders, diabetes, obesity, obesity-related conditions, nonalcoholic steatohepatitis (NASH), fatty liver disease, chronic kidney disease (CKD), polycystic ovary syndrome (PCOS), cardiovascular disease (CVD), obstructive sleep apnea (OSA), retinopathy, peripheral vascular disease (PVD), peripheral artery disease (PAD), and neuropathy (e.g., diabetic neuropathy). The methods herein described include combination administrations of a metabolic hormone and a glucagon-like peptide 1 receptor agonist (GLP-1 RA) that augment satiety induction and treatment of metabolic syndrome, diabetes, obesity, and obesity-related disorders.

In one aspect, the invention features a method of inducing satiety or treating a condition selected from metabolic syndrome, diabetes, obesity, an obesity-related disorder, NASH, fatty liver disease, CKD, PCOS, CVD, OSA, retinopathy, PVD, PAD, and neuropathy (e.g., diabetic neuropathy), the method including the step of topical-lingual administration of a dose of from 2.5 μg to 2.5 mg (e.g., from about 10 μg to about 1 mg, e.g., from about 25 μg to about 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of any one of Tables 1, 2, 3, 4, 5, 6, 7 or 8.

In some embodiments the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 2. In some embodiments, the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 3. In some embodiments, the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 4. In some embodiments, the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 5. In some embodiments, the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 6. In some embodiments, the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 7. In some embodiments, the method includes topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen of Table 8.

In some embodiments, the metabolic hormone targets a Y2 receptor of the subject. In some embodiments, the Y2 receptor is associated with a tongue of the subject.

In some embodiments, the metabolic hormone is Peptide YY (PYY), PYY(3-36), leptin, oxyntomodulin (OXM), cholecystokinin (CCK), insulin, amylin, gastric inhibitory peptide (GIP), glucagon, or analog, variant, or biologically active fragment of any of the above. In some embodiments, 2.5 μg, 10 μg, 25 μg, 50 μg, 100 μg, or 250 μg of the metabolic hormone is administered. In some embodiments, the metabolic hormone is formulated as an orally dissolvable tablet (ODT), a lozenge, a film, or a spray. In some embodiments, the ODT is in a rapidly dissolving form including partially hydrolyzed gelatin at a concentration of from about 1% to 6% w/v, mannitol, hydrolyzed dextran, alginate, polyvinyl alcohol, polyvinylpyrrolidone, acacia, aspartame, sodium methylparaben, sodium propylparaben, phenylalanine, water, or a combination thereof. In some embodiments, the method reduces a GLP-1-associated side effect. In some embodiments, the GLP-1-associated side effect includes nausea, vomiting, diarrhea, abdominal pain, constipation, pancreatitis, diabetic retinopathy complications, hypoglycemia, acute kidney injury, hypersensitivity, or a combination thereof. In some embodiments, the GLP-1 receptor agonist is formulated as a tablet, a gel capsule, or a liquid.

In another aspect, the invention features a method of inducing satiety or treating a condition selected from metabolic syndrome, diabetes, obesity, an obesity-related disorder, NASH, CKD, PCOS, CVD, OSA, retinopathy, PVD, PAD, and neuropathy disorder including topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject that has completed a systemic GLP-1 RA dose escalation regimen of Table 9.

In another aspect, the invention features a method of inducing satiety or treating a condition selected from metabolic syndrome, diabetes, obesity, an obesity-related disorder, NASH, CKD, PCOS, CVD, OSA, retinopathy, PVD, PAD, and neuropathy including topical-lingual administration of a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone to a subject under a GLP-1 RA treatment regimen wherein the GLP-1 RA is exenatide formulated for immediate release, and the treatment regimen includes systemically administering n*5 μg of exenatide, wherein n has a value of from 1-2, the GLP-1 RA is exenatide formulated for extended release, and the treatment regimen includes systemically administering n*2 mg of exenatide, wherein n has a value of 1, the GLP-1 RA is lixisenatide and the treatment regimen includes systemically administering n*10 μg of lixisenatide, wherein n has a value of from 1-2, the GLP-1 RA is liraglutide and the treatment regimen includes systemically administering n*600 μg of liraglutide, wherein n has a value of from 1-10, the GLP-1 RA is semaglutide and the treatment regimen includes systemically administering n*250 μg of semaglutide, wherein n has a value of from 1-100, the GLP-1 RA is dulaglutide and the treatment regimen includes systemically administering n*750 μg of dulaglutide, wherein n has a value of from 1-10, the GLP-1 RA is tirzepatide and tirzepatide treatment regimen A includes systemically administering n*4 mg, wherein n has a value of from 1-10, or the GLP-1 RA is tirzepatide and tirzepatide treatment regimen B includes systemically administering n*2.5 mg, wherein n has a value of from 1-10.

In some embodiments, the GLP-1 RA is semaglutide and the treatment regimen includes systemically administering n*1.7 μg of semaglutide, wherein n has a value of from 1-10, or the GLP-1 RA is semaglutide and the treatment regimen includes systemically administering n*2.4 μg of semaglutide, wherein n has a value of from 1-10.

In another aspect, the invention features a method of inducing satiety or treating a condition selected from metabolic syndrome, diabetes, obesity, an obesity-related disorder, NASH, CKD, PCOS, CVD, OSA, retinopathy, PVD, PAD, and neuropathy in a subject including systemic administration from 10% to 90% of a GLP-1 RA to the subject receiving a first dosage regimen of Table 9, and topical-lingual administration of a dose of from 2.5 μg to 250 μg of a metabolic hormone to the mouth of the subject.

In some embodiments, the method includes systemic administration of from 10% to 90% of a GLP-1 RA to the subject receiving a second dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone.

In some embodiments, the method includes systemic administration of from 10% to 90% of a GLP-1 RA to the subject receiving a third dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of 25 μg, 50 μg, 75 μg, 100 μg, 125 μg, 150 μg, 175 μg, 200 μg, 225 μg, or 250 μg) of a metabolic hormone.

In some embodiments, the method includes systemic administration of from 10% to 90% of a GLP-1 RA to the subject receiving a fourth dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of 25 μg, 50 μg, 75 μg, 100 μg, 125 μg, 150 μg, 175 μg, 200 μg, 225 μg, or 250 μg) of a metabolic hormone.

In some embodiments, the method includes systemic administration of from 10% to 90% of a GLP-1 RA to the subject receiving a fifth dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone.

In some embodiments, the method includes systemic administration of from 10% to 90% of a GLP-1 RA to the subject receiving a sixth dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone.

In some embodiments, the method includes systemic administration of from 10% to 90% of a GLP-1 RA to the subject receiving a seventh dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone.

In some embodiments, the method includes systemic administration of from 10% to 90% of a GLP-1 RA to the subject receiving an eighth dosage regimen of Table 9, and topical-lingual administration of a dose of 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) of a metabolic hormone.

In another aspect, the invention features a kit that includes a metabolic hormone formulated for topical-lingual administration in a dose of from 2.5 μg to 2.5 mg (e.g., from 1 μg to 1 mg, e.g., from 10 μg to 1 mg, e.g., from 2.5 μg to 250 μg, e.g., from 25 μg to 250 μg, e.g., a dose of about 25 μg, 50 μg, 75 μg, 100 μg, 125 μg, 150 μg, 175 μg, 200 μg, 225 μg, or 250 μg) and a dose of a GLP-1 RA formulated for systemic administration (e.g., as recited in any one of Tables 1-9).

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

The term “active ingredient,” as used herein, refers to a metabolic hormone, a GLP-1 RA, their analogs, variants, or biologically active fragments of the metabolic hormone that induces a biological response when administered to a subject.

The term “bind,” “binds,” or “binding” refers to an association between a metabolic hormone or a portion of the metabolic hormone, and a Y2 receptor through a chemical bond (e.g., ionic, covalent, or hydrophobic) or other chemical or physical attraction between the metabolic hormone or a portion thereof and a Y2 receptor where a biological response is induced by the association between metabolic hormone and the Y receptor. See e.g., Doods, Peptides. 16:1389-1394, 1995.

As used herein, the term “extended release,” in relation to any composition according to the invention refers to the release of all or part of the composition within 10 weeks of administration. A composition formulated for extended release may release an active ingredient in one or more phases of release.

The term “immediate release,” as used herein, in relation to any composition according to the invention refers to the release which is not modified release and releases all or part of the composition within 60 minutes of administration.

As used herein, “locally” or “local administration” means administration to a particular site of the body intended for a local effect and not a systemic effect. The terms “local,” or “local administration,” or variations thereof refer to administration of any of the active ingredients herein described with no substantial change in the level of the active ingredient in the blood of a subject.

The term “metabolic disorder,” as used herein, refers to a human or animal condition or disease associated with and/or resulting from abnormal function or control of the metabolic system (e.g., obesity, diabetes, fatty liver disease, NASH, PCOS, elevated blood glucose levels, CKD, CVD, OSA, retinopathy, PVD, PAD, and/or neuropathy (e.g., diabetic neuropathy)). The term “disorder” generally refers to disruption to regular bodily structure and function or a pathophysiological response to internal or external factors.

As used herein, the term “subject,” represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject.

The term “systemic administration,” as used herein, refers to the delivery of any composition according to the invention that increases blood or plasma levels of one or more active ingredients, its analogs, or variants, to above a limit of detection.

The term “therapeutic dose,” as used herein, refers to a dose at which a pharmaceutically active ingredient can treat, ameliorate, or reduce symptoms associated with a disease, disorder, or condition.

The term “treatment regimen,” as used herein, refers to at least the dose, frequency, and duration of any composition herein described being taken by a subject for treatment or prevention of any of the diseases or conditions herein described.

As used herein, the term “targets,” refers to an association between an active ingredient of any composition of the invention (e.g., a metabolic hormone or a GLP-1 RA) or a portion of the active ingredient and a receptor of the subject through a chemical bond, or other chemical or physical interaction where a biological response is induced by the association.

As used herein, the terms “topical-lingual administration,” “topical-lingually,” or variations thereof, refer to the local administration of a metabolic hormone to the epithelium of the mouth and/or tongue of a subject with substantially no change in the levels of metabolic hormone in the blood of the subject (e.g., substantially no systemic exposure).

### DETAILED DESCRIPTION OF THE INVENTION

In general, the invention features methods and kits for inducing satiety and treating a condition affecting metabolism, such as metabolic syndrome, diabetes, obesity, and obesity-related disorders. The invention is based, in part, upon the surprising discovery of a synergistic effect between the administration of a glucagon-like peptide 1 receptor agonist (GLP-1 RA) and a metabolic hormone, such as Peptide YY (PYY). The invention also provides methods and kits for treatment for subjects who have previously been administered a therapeutic regimen of a GLP-1 RA, but would benefit from the subsequent treatment with a metabolic hormone, or a combination therapy that includes a metabolic hormone and a GLP-1 RA. The methods and kits contemplated herein are described in more detail below.

### Compositions for Inducing Satiety and Treating Metabolic Disorders and Conditions Associated Therewith

### GLP-1 RA Compositions

The methods herein described include systemically administering a composition that includes a GLP-1 RA, or a biologically active fragment, variant, or analog thereof. In some embodiments, the GLP-1 RA is an approved therapeutic for inducing satiety and/or treating any of the herein described indications.

In some embodiments, the GLP-1 RA is exenatide, which contains a polypeptide having the sequence set forth in SEQ ID NO: 1. In some embodiments, the exenatide is formulated for immediate release (IR). In some embodiments, the exenatide is formulated for extended release (ER).

In some embodiments, the GLP-1 RA is lixisenatide, which contains a polypeptide having the sequence set forth in SEQ ID NO: 2.

In some embodiments, the GLP-1 RA is liraglutide, which contains a polypeptide having the sequence set forth in SEQ ID NO: 3 and has a palmitic acid moiety attached to a glutamate spacer, which is attached to the lysine at position 20.

In some embodiments, the GLP-1 RA is semaglutide, which contains a polypeptide having the sequence set forth in SEQ ID NO: 4 and has a α-aminoisobutyric acid at position 2 and a lysine derivative at position 20, which is acylated with a stearic diacid.

In some embodiments, the GLP-1 RA is dulaglutide, which contains a polypeptide having the sequence set forth in SEQ ID NO: 5.

In some embodiments, the GLP-1 RA is tirzepatide, which contains a polypeptide having the sequence set forth in SEQ ID NO: 6 and an α-aminoisobutyric acid at positions 2 and 13 and a modified lysine at position 20, which contains a C20 fatty diacid conjugated via a glutamate linker.

In some embodiments, the GLP-1 RA is GLP-1, or a variant, analog, or biologically active fragment thereof. In some embodiments, GLP-1 or variant, analog, or biologically active fragment thereof has at least 70% (e.g., at least 70%, 75%, 80%, 85%, 90%, 95%, 97%, 99%, or 100%) sequence identity to SEQ ID NO: 17. In some embodiments, GLP-1 has the sequence set forth in SEQ ID NO: 17.

The composition of a GLP-1 RA, as herein described (e.g., lixisenatide, liraglutide, semaglutide, dulaglutide, or tirzepatide, can include one or more pharmaceutically acceptable excipients (e.g., propylene glycol, potassium sorbate, l-arginine, edetate disodium, monosodium phosphate, and polysorbate 20). In some embodiments, propylene glycol is present in a concentration of about 100 mg/ml, l-arginine is present in a concentration of about 25 mg/ml, potassium sorbate is present in a concentration of about 2 mg/ml, edetate disodium is present in a concentration of about 1.2 mg/ml, sodium phosphate monobasic dihydrate is present in a concentration of about 7.8 mg/ml, and polysorbate is present in a concentration of about 5 mg/ml. Furthermore, the compositions described herein can include co-solvent stabilizers like propylene glycol or other suitable co-solvent stabilizers (e.g., lower molecular weight polyethylene glycols (PEG) such as PEG 200 and 400, glycerin, and ethanol. In some embodiments, compositions described herein include amino acid stabilizers like L-arginine or other suitable amino acid stabilizers (e.g., alanine, aspartic acid, glycine, lysine, proline, or methionine). In some embodiments, compositions described herein can include preservatives like potassium sorbate, or other suitable preservatives (e.g., ascorbic acid, benzyl alcohol, benzoic acid, citric acid, chlorobutanol, m-cresol, glutathione, methionine, methylparaben, propylparaben, sodium sulfite, parahydroxybenzoate esters (methylhydroxybenzoate and propylhydroxybenzoate), boric acid and borate salts, sorbic acid and other sorbate salts besides potassium, and phenolics). compositions described herein can include antioxidants such as edetate disodium or another suitable antioxidant (e.g., sodium formaldehyde sulphoxylate, butylated hydroxyanisole, and butylated hydroxytoluene). In some embodiments, the compositions described herein include buffers (e.g., acetate, carbonate, citrate, citrate-phosphate, glycine, HEPES, histidine, maleate, phosphate, succinate, tartrate, and triethanolamine (Tris)). In some embodiments, the compositions described herein can include surfactants, such as polysorbate 20 or other suitable surfactants (e.g., Poloxamer 188/407, polysorbate 40 or 80, or sodium lauryl sulfate).

### GLP-1 RA Routes of Administration

The compositions of the invention including a GLP-1 RA can be formulated to systemically deliver the GLP-1 RA. Compositions may be formulated for any suitable route and means of administration. Pharmaceutically acceptable carriers or diluents include those used in formulations suitable for oral, rectal, nasal, or parenteral (including subcutaneous, intramuscular, intravenous, intradermal, and transdermal) administration. The formulations may conveniently be presented in unit dosage form and may be prepared by any of the methods well known in the art of pharmacy. Subcutaneous or transdermal modes of administration may be particularly suitable for certain of the compounds described herein. In some embodiments, the GLP-1 RA is formulated as a tablet, a gel capsule, or a liquid. The compositions of the invention may be in unit dosage form. In such form, the composition is divided into unit doses containing appropriate quantities of the active ingredient (e.g., exenatide IR). The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of an injection pen.

### Metabolic Hormone Compositions

The methods herein described include topical-lingual administration of a metabolic hormone, or a biologically active fragment, variant, or analog thereof. The metabolic hormone targets (e.g., binds, associates, or interacts with) Y2 receptors of the oral cavity of a subject. In some embodiments, the metabolic hormone targets Y2 receptors expressed on the tongue of the subject. In some embodiments, the metabolic hormone is Peptide YY (PYY), PYY(3-36), leptin, oxyntomodulin, cholecystokinin, insulin, amylin, gastric inhibitory peptide, or glucagon.

In some embodiments, the metabolic hormone is PYY, or an analog, variant, or biologically active fragment thereof (e.g., (Pro34)PYY, PYY analog NNC0165-1273, PYY analog NNC0165-1875, or PYY analog NNC0165-1562). In some embodiments, PYY or variant, analog, or biologically active fragment thereof has at least 70% (e.g., 70%, 75%, 80%, 85%, 90%, 95%, 97%, 99%, or 100%) sequence identity to SEQ ID NO: 7. In some embodiments, PYY has the amino acid sequence set forth in SEQ ID NO: 7. In some embodiments, the methods herein described include the topical-lingual administration of PYY or a variant, analog, or biologically active fragment thereof in a dose of from about 2.5 μg to about 2.5 mg. In some embodiments, the methods herein described include the topical-lingual administration of PYY or a variant, analog, or biologically active fragment thereof in a dose of from about 10 μg to about 1 mg. In some embodiments, the methods herein described include the topical-lingual administration of PYY or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg). In some embodiments, the PYY variant is [Pro34]PYY. In some embodiments, [Pro34]PYY or variant, analog, or biologically active fragment thereof has at least 70% (e.g., at least 70%, 75%, 80%, 85%, 90%, 95%, 97%, 99%, or 100%) sequence identity to SEQ ID NO: 8. In some embodiments, [Pro34]PYY has the amino acid sequence set forth in SEQ ID NO: 8. In some embodiments, the PYY variant is NNC065-1273, which contains the PYY(3-36) polypeptide with a beta-homo-arginine at position 35. In some embodiments, the PYY variant is NNC0165-1875. In some embodiments, the PYY variant is NNC0165-1562. In some embodiments, the PYY analog or variant is described, e.g., in Lear et al. J. of Med. Chem. 63:9660-9671, 2020, which is hereby incorporated by reference in its entirety. In some embodiments, the PYY analog is PYY-Ab (PYY conjugated to an antibody or an Fc region of an antibody) or PYY conjugated to one or more PEG moieties, e.g., as described in Rangwala et al. Cell Metab. 29:837-843, 2019, which is hereby incorporated by reference in its entirety. In some embodiments, the PYY variant or analog is any variant described, e.g., in U.S. Pat. No. 8,217,001, the disclosure of which is hereby incorporated by reference in its entirety.

In some embodiments, the metabolic hormone is PYY(3-36), or an analog, variant, or biologically active fragment thereof. In some embodiments, PYY(3-36) variant, analog, or biologically active fragment thereof has at least 70% (e.g., at least 70%, 75%, 80%, 85%, 90%, 95%, 97%, 99%, or 100%) sequence identity to SEQ ID NO: 9. In some embodiments, PYY(3-36) has the amino acid sequence set forth in SEQ ID NO: 9.

In some embodiments, the PYY(3-36) variant, analog, or biologically active fragment thereof is PYY(26-36), PYY(25-36), PYY(24-36), PYY(23-36), PYY(22-36), PYY(21-36), PYY(20-36), PYY(19-36), PYY(18-36), PYY(17-36), PYY(16-36), PYY(15-36), PYY(14-36), PYY(13-36), PYY(12-36), PYY(11-36), PYY(10-36), PYY(9-36), PYY(8-36), PYY(7-36), PYY(6-36), PYY(5-36), or PYY(4-36), as in Balasubramaniam et al., Pept Res 1:32-35, 1998; Liu et al., J. Gastrointest Surg. 5:147-152, 2001, herein incorporated by reference in their entirety. In some embodiments, the PYY(3-36) variant, analog, or fragment thereof may be a fragment with a single point mutation e.g., single point mutation of PYY(25-36) such as [Lys²⁵]PPY(25-36), [Thr²⁷]PPY(25-36), [Phe²¹]PPY(25-36), [Ile²⁸]PYY(25-36), [Val²⁸]PYY(25-36), [Gln²⁹]PYY(25-36), [Ile³⁰]PYY(25-36), [Val³⁰]PYY(25-36), [Ile³¹]PYY(25-36), [Leu³¹]PYY(25-36), [Ser³²]PYY(25-36), [Lys³³]PYY(25-36), [Asn³⁴]PYY(25-36), [Lys³⁵]PYY(25-36), [Thr³⁶]PYY(25-36), or [Phe³⁶]PYY(25-36) or a single point mutation of PYY(24-36) such as [Ile²⁴]PYY(24-36) or [Val24]PYY(24-36). In some embodiments, the PYY(3-36) variant, analog, or biologically active fragment thereof may be a fragment with a double point mutation e.g., double point mutation of PYY(25-36) such as [Lys25, Thr27]PPY(25-36), [Lys25, Phe27]PPY(25-36), [Lys25, Ile28]PPY(25-36), [Lys25, Val28]PPY(25-36), [Lys25, Gln29]PPY(25-36), [Lys²⁵, Ile³⁰]PPY(25-36), [Lys²⁵, Val³⁰]PPY(25-36), [Lys²⁵, Ile³¹]PPY(25-36), [Lys²⁵, Leu³¹]PPY(25-36), [Lys²⁵, Ser³²]PPY(25-36), [Lys²⁵, Lys³³]PPY(25-36), [Lys²⁵, Asn³⁴]PPY(25-36), [Lys²⁵, Lys³⁵]PPY(25-36), [Lys²⁵, Thr³⁶]PPY(25-36), [Lys²⁵, Phe³⁶]PPY(25-36), [Thr²⁷, Ile²⁸]PPY(25-36), [Thr²⁷, Val²⁸]PPY(25-36), [Thr²⁷, Gln²⁹]PPY(25-36), [Thr²⁷, Ile³⁰]PPY(25-36), [Thr²⁷, Val³⁰]PPY(25-36), [Thr²⁷, Ile³¹]PPY(25-36), [Thr²⁷, Leu³¹]PPY(25-36), [Thr²⁷, Ser³²]PPY(25-36), [Thr²⁷, Lys³³]PPY(25-36), [Thr²⁷, Asn³⁴]PPY(25-36), [Thr²⁷, Lys³⁵]PPY(25-36), [Thr²⁷, Thr³⁶]PPY(25-36), [Thr²⁷, Phe³⁶]PPY(25-36), [Phe²⁷, Ile²⁸]PPY(25-36), [Phe²⁷, Val²⁸]PPY(25-36), [Phe²⁷, Gln²⁹]PPY(25-36), [Phe²⁷, Ile³⁰]PPY(25-36), [Phe²⁷, Val³⁰]PPY(25-36), [Phe²⁷, Ie³¹]PPY(25-36), [Phe²⁷, Leu³¹]PPY(25-36), [Phe²⁷, Ser³²]PPY(25-36), [Phe²⁷, Lys³³]PPY(25-36), [Phe²⁷, Asn³⁴]PPY(25-36), [Phe²⁷, Lys³⁵]PPY(25-36), [Phe²⁷, Thr³⁶]PPY(25-36), [Phe²⁷, Phe³⁶]PPY(25-36), [Gln²⁹, Ile³⁰]PYY(25-36), [Gln²⁹, Val³⁰]PYY(25-36), [Gln²⁹, Ile³¹]PYY(25-36), [Gln²⁹, Leu³¹]PYY(25-36), [Gln²⁹, Ser³²]PYY(25-36), [Gln²⁹, Leu³³]PYY(25-36), [Gln²⁹, Asn³⁴]PYY(25-36), [Gln²⁹, Leu³³]PYY(25-36), [Gln²⁹, Thr³⁶]PYY(25-36), [Gln²⁹, Phe³⁰]PYY(25-36), [Ile³⁰, Ile³¹]PYY(25-36), [Ile³⁰, Leu³¹]PYY(25-36), [Ile³⁰, Ser³²]PYY(25-36), [Ile³⁰, Lys³³]PYY(25-36), [Ile³⁰, Asn³⁴]PYY(25-36), [Ile³⁰, Lys³³]PYY(25-36), [Ile³⁰, Thr³⁰]PYY(25-36), [Ile³⁰, Phe³⁰]PYY(25-36), [Val³⁰, Ile³¹]PYY(25-36), [Val³⁰, Leu³¹]PYY(25-36), [Val³⁰, Ser³²]PYY(25-36), [Val³⁰, Lys³³]PYY(25-36), [Val³⁰, Asn³⁴]PYY(25-36), [Val³⁰, Lys³⁵]PYY(25-36), [Val³⁰, Thr³⁰]PYY(25-36), [Val³⁰, Phe³⁰]PYY(25-36), [Ile³¹, Ser³²]PYY(25-36), [Ile³¹, Lys³³]PYY(25-36), [Ile³¹, Asn³⁴]PYY(25-36), [Ile³¹, Lys³³]PYY(25-36), [Ile³¹, Thr³⁰]PYY(25-36), [Leu³¹, Phe³⁰]PYY(25-36), [Leu³¹, Ser³²]PYY(25-36), [Val³¹, Lys³³]PYY(25-36), [Leu³¹, Asn³⁴]PYY(25-36), [Leu³¹, Lys³³]PYY(25-36), [Leu³¹, Thr³⁰]PYY(25-36), [Leu³¹, Phe³⁰]PYY(25-36), [Ser³², Lys³³]PYY(25-36), [Ser³², Asn³⁴]PYY(25-36), [Ser³², Lys³⁵]PYY(25-36), [Ser³², Thr³⁶]PYY(25-36), [Ser³², Phe³⁶]PYY(25-36), [Lys³³, Asn³⁴]PYY(25-36), [Lys³³, Lys³⁵]PYY(25-36), [Lys³³, Thr³⁶]PYY(25-36), [Lys³³, Phe³⁶]PYY(25-36), [Asn³⁴, Lys³⁵]PYY(25-36), [Asn³⁴, Thr³⁶]PYY(25-36), [Asn³⁴, Phe³⁶]PYY(25-36), [Lys³⁵, Thr³⁶]PYY(25-36), or [Lys³⁵, Phe³⁶]PYY(25-36).

In some embodiments, the methods herein described include the topical-lingual administration of PYY(3-36) or a variant, analog, or biologically active fragment thereof in a dose of from about 2.5 μg to about 2.5 mg. In some embodiments, the methods herein described include the topical-lingual administration of PYY(3-36) in a dose of from about 10 μg to about 1 mg. In some embodiments, the methods herein described include the topical-lingual administration of PYY(3-36) in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is leptin, or an analog, variant, or biologically active fragment thereof. In some embodiments, the leptin analog is a leptin receptor antagonist as in Peters J. H., et al, Endocrinology, 148(6):2878-85, 2007, herein incorporated by reference in its entirety. In some embodiments, leptin or a variant, analog, or biologically active fragment thereof has at least 70% (e.g., at least 70%, 75%, 80%, 85%, 90%, 95%, 97%, 99%, or 100%) sequence identity to SEQ ID NO: 10. In some embodiments, leptin has the amino acid sequence set forth in SEQ ID NO: 10. In some embodiments, the analog of leptin is the recombinant analog metreleptin (MYALEPT®), which contains a polypeptide having the sequence set forth in SEQ ID NO: 18 and contains a disulfide bridge connecting amino acid residues 97 and 147. In some embodiments, the methods herein described include the topical-lingual administration of leptin or a variant, analog, or biologically active fragment thereof in a dose of from about 2.5 μg to about 2.5 mg. In some embodiments, the methods herein described include the topical-lingual administration of leptin or a variant, analog, or biologically active fragment thereof in a dose of from about 10 μg to about 1 mg. In some embodiments, the methods herein described include the topical-lingual administration of leptin or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is OXM, or an analog, variant, or biologically active fragment thereof. In some embodiments, OXM or a variant, analog, or biologically active fragment thereof has at least 70% (e.g., at least 70%, 75%, 80%, 85%, 90%, 95%, 97%, 99%, or 100%) sequence identity to SEQ ID NO: 11. In some embodiments, OXM has the amino acid sequence set forth in SEQ ID NO: 11. In some embodiments, the analog of OXM is the synthetic analog OXM6421 synthesized for long-acting effect as described in Liu, Y-L., Int. J. Obes., 2010, 34(12):1715-25 herein incorporated by reference in its entirety. In some embodiments, the analog of OXM is the long-acting polyethylene glycol (PEG)-OXM functionalized with high molecular weight PEG molecules as described in Bianchi, E., Bioorg. Med. Chem., 2013, 21(22):7064-73 herein incorporated by reference in its entirety. In some embodiments, the analog of OXM is the sustained release analog OX-SR as described in Scott, R., Peptides, 2018, 104:70-77 herein incorporated by reference in its entirety. In some embodiments, the methods herein described include the topical-lingual administration of OXM or a variant, analog, or biologically active fragment thereof in a dose of from about 2.5 μg to about 2.5 mg. In some embodiments, the methods herein described include the topical-lingual administration of OXM or a variant, analog, or biologically active fragment thereof in a dose of from about 10 μg to about 1 mg. In some embodiments, the methods herein described include the topical-lingual administration of OXM or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is CCK, or an analog, variant, or biologically active fragment thereof. In some embodiments, CCK or variant, analog, or biologically active fragment thereof has at least 70% (e.g., at least 70%, 75%, 80%, 85%, 90%, 95%, 97%, 99%, or 100%) sequence identity to SEQ ID NO: 12. In some embodiments, CCK has the amino acid sequence set forth in SEQ ID NO: 12. In some embodiments, the analog of CCK is caerulein as described in Sporti J. Surg. Oncol., 54:11-16, 1994, herein incorporated by reference in its entirety. In some embodiments, the analog of CCK is the synthetic analog Thr28NIE31CCK25-33 (CCK9) as described in Mössner, Z. Gastroenterol., 29:59-64, 1991, herein incorporated by reference in its entirety. In some embodiments, the methods herein described include the topical-lingual administration of CCK or a variant, analog, or biologically active fragment thereof in a dose of from about 2.5 μg to about 2.5 mg. In some embodiments, the methods herein described include the topical-lingual administration of CCK or a variant, analog, or biologically active fragment thereof in a dose of from about 10 μg to about 1 mg. In some embodiments, the methods herein described include the topical-lingual administration of CCK or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is insulin, or an analog (e.g., insulin aspart (NOVOLOG®), insulin glargine (LANTUS®), insulin lispro (LYUMJEV™), insulin glulisine (APIDRA®), or insulin detemir (LEVEMIR®), insulin degludec (TRESIBA®), NPH insulin (HUMULIN® N or NOVOLIN® N), a variant, or biologically active fragment thereof. In some embodiments, insulin or a variant, analog, or biologically active fragment thereof has at least 70% (e.g., at least 70%, 75%, 80%, 85%, 90%, 95%, 97%, 99%, or 100%) sequence identity to SEQ ID NO: 13. In some embodiments, insulin has the amino acid sequence set forth in SEQ ID NO: 13. In some embodiments, the analog of insulin is insulin aspart (NOVOLOG®), having an A chain with the sequence set forth in SEQ ID NO: 19 and a B chain with the sequence set forth in SEQ ID NO: 20. In some embodiments, the analog of insulin is insulin glargine (LANTUS®), having an A chain with sequence set forth in SEQ ID NO: 21 and a B chain with to the sequence set forth in SEQ ID NO: 22. In some embodiments, the analog of insulin is insulin lispro (LYUMJEV™), having an A chain with the sequence set forth in SEQ ID NO: 23 and a B chain with to the sequence set forth in SEQ ID NO: 24. In some embodiments, the analog of insulin is insulin glulisine (APIDRA®), having an A chain with the sequence set forth in SEQ ID NO: 25 and a B chain with to the sequence set forth in SEQ ID NO: 26. In some embodiments, the analog of insulin is insulin detemir (LEVEMIR®), having an A chain with to the sequence set forth in SEQ ID NO: 27 and a B chain with to the sequence set forth in SEQ ID NO: 28. In some embodiments, the methods herein described include the topical-lingual administration of insulin or a variant, analog, or biologically active fragment thereof in a dose of from about 2.5 μg to about 2.5 mg. In some embodiments, the methods herein described include the topical-lingual administration of insulin or a variant, analog, or biologically active fragment thereof in a dose of from about 10 μg to about 1 mg. In some embodiments, the methods herein described include the topical-lingual administration of insulin or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is amylin, or an analog (e.g., pramlintide (SYMLIN®)), variant, or biologically active fragment thereof. In some embodiments, amylin or a variant, analog, or biologically active fragment thereof has at least 70% (e.g., at least 70%, 75%, 80%, 85%, 90%, 95%, 97%, 99%, or 100%) sequence identity to SEQ ID NO: 14. In some embodiments, amylin has the amino acid sequence set forth in SEQ ID NO: 14. In some embodiments, the analog of amylin is pramlintide (SYMLIN®), which contains a polypeptide having to the sequence set forth in SEQ ID NO: 29. In some embodiments, the methods herein described include the topical-lingual administration of amylin or a variant, analog, or biologically active fragment thereof in a dose of from about 2.5 μg to about 2.5 mg. In some embodiments, the methods herein described include the topical-lingual administration of amylin or a variant, analog, or biologically active fragment thereof in a dose of from about 10 μg to about 1 mg. In some embodiments, the methods herein described include the topical-lingual administration of amylin or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is GIP, or an analog (e.g., D-GIP₁₋₃₀, N-AcGIP(LysPAL16, or N-AcGIP(LysPAL37), variant, or biologically active fragment thereof. In some embodiments, GIP or a variant, analog, or biologically active fragment thereof has at least 70% (e.g., at least 70%, 75%, 80%, 85%, 90%, 95%, 97%, 99%, or 100%) sequence identity to SEQ ID NO: 15. In some embodiments, GIP has the amino acid sequence set forth in SEQ ID NO: 15. In some embodiments, the analog of GIP is D-GIP₁₋₃₀, N-AcGIP(LysPAL16, or N-AcGIP(LysPAL37) as described in Sekar R., Int. Rev. Cell Mol. Biol., 2016, 326279-341 herein incorporated by reference in its entirety. In some embodiments, the methods herein described include the topical-lingual administration of GIP in a dose of from about 2.5 μg to about 2.5 mg. In some embodiments, the methods herein described include the topical-lingual administration of GIP in a dose of from about 10 μg to about 1 mg. In some embodiments, the methods herein described include the topical-lingual administration of GIP in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the metabolic hormone is glucagon, or an analog (e.g., dasiglucagon (ZEGALOGUE®), AB-G023, or HM15136), variant, or biologically active fragment thereof. In some embodiments, glucagon or a variant, analog, or biologically active fragment thereof has at least 70% (e.g., 70%, 75%, 80%, 85%, 90%, 95%, 97%, 99%, or 100%) sequence identity to SEQ ID NO: 16. In some embodiments, glucagon has the amino acid sequence set forth in SEQ ID NO: 16. In some embodiments, the analog of glucagon is dasiglucagon (ZEGALOGUE®), which contains a polypeptide having to the sequence set forth in SEQ ID NO: 30 and an α-aminoisobutyric acid at position 16. In some embodiments, the analog of glucagon is AB-G023, a solution stable, soluble glucagon analog. In some embodiments, the analog of glucagon is HM15136, a long-acting Glucagon analog chemically conjugated with constant region of human immunoglobulin via non-peptidyl flexible linker. In some embodiments, the methods herein described include the topical-lingual administration of glucagon or a variant, analog, or biologically active fragment thereof in a dose of from about 2.5 μg to about 2.5 mg. In some embodiments, the methods herein described include the topical-lingual administration of glucagon or a variant, analog, or biologically active fragment thereof in a dose of from about 10 μg to about 1 mg. In some embodiments, the methods herein described include the topical-lingual administration of glucagon or a variant, analog, or biologically active fragment thereof in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the compositions described herein are adapted for delivery to the oral cavity (e.g., intraoral, oromucosal, transmucosal, topical lingual, gargles, mouthwashes, gingival solutions, oromucosal solutions and oromucosal suspensions, semi-solid oromucosal preparations (including for example gingival gel, gingival paste, oromucosal gel, oromucosal paste)), oromucosal drops, oromucosal sprays and sublingual sprays (including oropharyngeal sprays), dry powder sprays, lozenges and pastilles, compressed lozenges, sublingual tablets and buccal tablets, oromucosal capsules, mucoadhesive preparations)). See, e.g., Oromucosal Preparations, (Ph Eur monograph 1807). In some embodiments, the metabolic hormone in the pharmaceutical composition is adapted for binding to the Y2 receptors expressed in the oral cavity (e.g., on the tongue).

In some embodiments, the metabolic hormone is formulated as a lozenge, a film, a spray, or an orally dissolvable tablet (ODT). In some embodiments, the metabolic hormone is formulated as an ODT using a formulation that rapidly dissolves on the tongue of a subject. For example, the formulation may have partially hydrolyzed gelatin at a concentration of from about 1% to 6% w/v (e.g., about 1%, about 2%, about 3%, about 4%, about 5%, or about 6%), mannitol, hydrolyzed dextran, alginate, polyvinyl alcohol, polyvinylpyrrolidone, acacia, aspartame, sodium methylparaben, sodium propylparaben, phenylalanine, water, or a combination thereof. In some embodiments, the ODT is formulated using the ZYDIS® formulation, as in U.S. Pat. Nos. 4,305,502, 4,371,516, and 5,738,875, herein incorporated in their entirety by reference.

In some embodiments, the compositions include excipients that increase the time the metabolic hormone (e.g., PYY(3-36)) is in contact with the mucosa (e.g., viscosity enhancement, encapsulation, and controlled release). Without being bound by theory, it is believed that increasing the contact time of the pharmaceutical formulation with the mucosa, leads to increased binding of the metabolic hormone to its receptor on the tongue. Suitable excipients for viscosity enhancement include rheology modifiers which also may be mucoadhesive such as methylcellulose, hydroxyethylcellulose, hydroxypropylcellulose, alginic acid, polyvinylpyrrolidone, and sodium carboxymethylcellulose. Suitable excipients for modified release of the metabolic hormone in the oral cavity include mucoadhesive permeation enhancers such as 23-lauryl ether, aprotinin, azone, benzalkonium chloride, cetylpyridinium chloride, cetyltrimethylammonium bromide, cyclodextrins, dextran sulfate, and lauric acid. Other suitable mucoadhesive polymers used for buccal or intraoral delivery include agarose, chitosan, gelatin, hyaluronic acid, gums (e.g. guar, hakea, xanthan, gellan, carrageenan, pectin, and sodium alginate), cellulose derivatives (e.g., CMC, thiolated CMC, sodium CMC, HEC, HPMC, MC, methylhydroxylethylcellulose), poly (acrylic acid)-based polymers (e.g., CP, PC, PAA, polyacrylates, poly(methylvinylether-co-methacrylic acid), poly(2-hydroxyethyl methacryalate), poly(alkylcyanoacryalate), poly(isohexylcyanocrylate), poly(isobutylcyanoacrylate), copolymer of acrylic acid and PEG, poly(N-2-hydroxypropyl methacrylamide), PHPMAm, polyoxyethylene, PVA, PVP, and other thiolated polymers; scleroglucan, PVA, steroidal detergents, non-ionic surfactants, laureth-9, sodium fusidate, included sodium lauryl, sodium laurate (e.g., pH 8.9), palmitoyl carnitine, lauric acid/propylene glycol vehicle, Brij 78, sodium deoxycholate, sodium lauryl sulfate, lecithin and PVP. See, e.g., International Journal of Pharmaceutics, Volume 53, Issue 3, 1 Aug. 1989, Pages 227-235.

In some embodiments, the pharmaceutical compositions include excipients that increase the residence time of a metabolic hormone in the saliva (e.g., the amount of time the metabolic hormone remains in the saliva without significant degradation of the peptide). Without being bound by theory, it is believed that increasing the residence time of the metabolic hormone in the saliva increases the opportunity for the metabolic hormone to bind its receptor on the tongue. The residence time in the saliva can optionally be adjusted to avoid increasing systemic exposure to the metabolic hormone through, for example, swallowing.

The composition of a metabolic hormone, as herein described, can include one or more pharmaceutically acceptable excipients (e.g., propylene glycol, potassium sorbate, l-arginine, edetate disodium, monosodium phosphate, and polysorbate 20). In some embodiments, propylene glycol is present in a concentration of about 100 mg/ml, l-arginine is present in a concentration of about 25 mg/ml, potassium sorbate is present in a concentration of about 2 mg/ml, edetate disodium is present in a concentration of about 1.2 mg/ml, sodium phosphate monobasic dihydrate is present in a concentration of about 7.8 mg/ml, and polysorbate is present in a concentration of about 5 mg/ml. Furthermore, the compositions described herein can include co-solvent stabilizers like propylene glycol or other suitable co-solvent stabilizers (e.g., lower molecular weight polyethylene glycols (PEG) such as PEG 200 and 400, glycerin, and ethanol. In some embodiments, compositions described herein include amino acid stabilizers like L-arginine or other suitable amino acid stabilizers (e.g., alanine, aspartic acid, glycine, lysine, proline, or methionine). In some embodiments, compositions described herein can include preservatives like potassium sorbate, or other suitable preservatives (e.g., ascorbic acid, benzyl alcohol, benzoic acid, citric acid, chlorobutanol, m-cresol, glutathione, methionine, methylparaben, propylparaben, sodium sulfite, parahydroxybenzoate esters (methylhydroxybenzoate and propylhydroxybenzoate), boric acid and borate salts, sorbic acid and other sorbate salts besides potassium, and phenolics). compositions described herein can include antioxidants such as edetate disodium or another suitable antioxidant (e.g., sodium formaldehyde sulphoxylate, butylated hydroxyanisole, and butylated hydroxytoluene). In some embodiments, the compositions described herein include buffers (e.g., acetate, carbonate, citrate, citrate-phosphate, glycine, HEPES, histidine, maleate, phosphate, succinate, tartrate, and triethanolamine (Tris)). In some embodiments, the compositions described herein can include surfactants like polysorbate 20 or other suitable surfactants (e.g., Poloxamer 188/407, polysorbate 40 or 80, or sodium lauryl sulfate).

In some embodiments, the excipients include flavorings to increase compliance with ingesting the composition. For example, the flavorings can be used to mask bitter or other undesirable flavor properties, or to make the composition compatible with the flavor of food that may be ingested before or after administration of the composition. Compatible flavorings include, for example, apple, banana, bubblegum, cherry, chocolate, grape, lemon, mango, orange, raspberry, strawberry, vanilla, watermelon, mint or a combination of the above flavors. In another aspect, these flavorings are dye-free, sugar-free, hypoallergenic, gluten-free, and casein-free.

### Metabolic Hormone Routes of Administration

The metabolic hormones disclosed herein are administered locally to the tongue of the subject. The metabolic hormone compositions herein described are formulated to topically deliver the metabolic hormone to an oral tissue, e.g., via topical-lingual delivery. Topical delivery of the metabolic hormone may provide beneficial outcomes, such as binding to a Y2 receptor of the oral tissue (e.g., Y2 receptors of the tongue) and transmitting a signal to the brain via the Y2 receptor, and/or avoiding systemic exposure to the metabolic hormone and any unwanted side effects of systemic exposure. Additionally, administration of the metabolic hormone locally to the Y2 receptors of the mouth induce satiety using doses substantially lower than doses required to achieve similar satiety induction results via systemic administration of metabolic hormones.

### Methods of Inducing Satiety and Treating Metabolic Disorders Using Combination Therapy

### Indications

The methods herein described include the administration of a metabolic hormone as part of a combination therapy with at least one GLP-1 RA for the induction of satiety and/or treatment of metabolic syndrome, diabetes, obesity, or any obesity-related disorder.

In some embodiments, the subject in need of the combination therapy has been diagnosed with obesity, an obesity-related disorder, metabolic syndrome, diabetes, dyslipidemia, hypertension, weight loss, weight gain, age-related weight gain, pediatric weight loss, NASH, CKD, PCOS, CVD, OSA, retinopathy, PVD, PAD, and neuropathy (e.g., diabetic neuropathy), and prevention of peripheral vascular degeneration, which can often require amputation. In some embodiments, the methods are used for maintenance of weight or prevention of weight gain.

### GLP-1 RA Treatment Regimen

In some embodiments, the subject is in the process of receiving a treatment regimen of GLP-1 RA. In some embodiments, the subject is in the process of receiving treatment with the maximum recommended dose (e.g., dosing based on body weight, age, or other clinical parameters). In some embodiments, the subject has completed a dosing escalation (Tables 1-9) of a GLP-1 RA and has a steady state level of GLP-1 RA in the blood or plasma. In some embodiments, the subject has started a GLP-1 RA treatment regimen or a dose escalation and has not reached the maximum recommended GLP-1 RA dose. In some embodiments, the subject has not been diagnosed with any of the previous indications but is at an elevated risk of developing any of the previous indications and/or has begun or completed a dose escalation with a GLP-1 RA prophylactically.

The methods herein described combine systemic administration of a GLP-1 RA with local administration (e.g., topical-lingual) of a metabolic hormone. In some embodiments, the subject is in the process of a dose escalation regimen of a GLP-1 RA (as in any one of Tables 1-9). In some embodiments, the subject is in the process of receiving a first dose of any one of the GLP-1 RAs in Table 1. In some embodiments, the subject is in the process of receiving a second dose of any one of the GLP-1 RAs in Table 2. In some embodiments, the subject is in the process of receiving a third dose of any one of the GLP-1 RAs in Table 3. In some embodiments, the subject is in the process of receiving a fourth dose of any one of the GLP-1 RAs in Table 4. In some embodiments, the subject is in the process of receiving a fifth dose of any one of the GLP-1 RAs in Table 5. In some embodiments, the subject is in the process of receiving a sixth dose of any one of the GLP-1 RAs in Table 6. In some embodiments, the subject is in the process of receiving a seventh dose of any one of the GLP-1 RAs in Table 7. In some embodiments, the subject is in the process of receiving an eighth dose of any one of the GLP-1 RAs in Table 8.

TABLE 1
First treatment of dose escalation regimen

| | | | | | | Route of |
|---|---|---|---|---|---|---|
| GLP-1 RA | Dose (mg) | Frequency | Duration | | Formulation | administration |
| Exenatide | 0.005 | Twice daily | 4 | weeks | Immediate | Systemic |
| | | | | | release (IR) | |
| Exenatide | 2 | Once weekly | no | | Extended | Systemic |
| (BYDURION ®) | | | escalation | | release (ER) | |
| | | | necessary | | | |
| Lixisenatide | 0.010 | Once daily | 14 | days | | Systemic |
| Liraglutide | 0.600 | Once daily | 7 | days | | Systemic |
| Semaglutide | 0.250 | Once weekly | 4 | weeks | | Systemic |
| Dulaglutide | 0.750 | Once weekly | 4 | weeks | | Systemic |
| Tirzepatide | 4 | Once weekly | 4 | weeks | | Systemic |
| Dose Regimen | | | | | | |
| A | | | | | | |
| Tirzepatide | 2.5 | Once weekly | 4 | weeks | | Systemic |
| Dose Regimen | | | | | | |
| B | | | | | | |

TABLE 2
Second treatment of dose escalation regimen

| | | | | | | Route of |
|---|---|---|---|---|---|---|
| | Dose | | | | Formula- | adminis- |
| GLP-1 RA | (mg) | Frequency | Duration | | tion | tration |
| Exenatide | 0.01 | Twice daily | 4 | weeks | Immediate | Systemic |
| | | | | | release | |
| Lixisenatide | 0.020 | Once daily | 14 | days | | Systemic |
| Liraglutide | 1.2 | Once daily | 7 | days | | Systemic |
| Semaglutide | 0.5 | Once weekly | 4 | weeks | | Systemic |
| Dulaglutide | 1.5 | Once weekly | 4 | weeks | | Systemic |
| Tirzepatide | 8 | Once weekly | 4 | weeks | | Systemic |
| Dose | | | | | | |
| Regimen A | | | | | | |
| Tirzepatide | 5 | Once weekly | 4 | weeks | | Systemic |
| Dose | | | | | | |
| Regimen B | | | | | | |

TABLE 3
Third treatment of dose escalation regimen

| | Dose | | | Route of |
|---|---|---|---|---|
| GLP-1 RA | (mg) | Frequency | Duration | administration |
| Liraglutide | 1.8 | Once daily | 7 days | Systemic |
| Semaglutide | 1 | Once weekly | 4 weeks | Systemic |
| Dulaglutide | 3 | Once weekly | 4 weeks | Systemic |
| Tirzepatide Dose | 12 | Once weekly | 4 weeks | Systemic |
| Regimen A | | | | |
| Tirzepatide Dose | 10 | Once weekly | 4 weeks | Systemic |
| Regimen B | | | | |

TABLE 4
Fourth treatment of dose escalation regimen

| | | | | Route of |
|---|---|---|---|---|
| GLP-1 RA | Dose (mg) | Frequency | Duration | administration |
| Liraglutide | 2.4 | Once daily | 7 days | Systemic |
| Semaglutide | 1.7 | Once weekly | 4 weeks | Systemic |
| Dulaglutide | 4.5 | Once weekly | 4 weeks | Systemic |
| Tirzepatide | 15 | Once weekly | 4 weeks | Systemic |
| Dose Regimen B | | | | |

TABLE 5
Fifth treatment of dose escalation regimen

| | | | | Route of |
|---|---|---|---|---|
| GLP-1 RA | Dose (mg) | Frequency | Duration | administration |
| Liraglutide | 3 | Once daily | 7 days | Systemic |
| Semaglutide | 2.4 | Once weekly | 4 weeks | Systemic |

TABLE 6
Sixth treatment of dose escalation regimen

| | | | | Route of |
|---|---|---|---|---|
| GLP-1 RA | Dose (mg) | Frequency | Duration | administration |
| Semaglutide | 3 | Once weekly | 4 weeks | Systemic |

TABLE 7
Seventh treatment of dose escalation regimen

| | | | | Route of |
|---|---|---|---|---|
| GLP-1 RA | Dose (mg) | Frequency | Duration | administration |
| Semaglutide | 7 | Once weekly | 4 weeks | Systemic |

TABLE 8
Eighth treatment of dose escalation regimen

| | | | | Route of |
|---|---|---|---|---|
| GLP-1 RA | Dose (mg) | Frequency | Duration | administration |
| Semaglutide | 14 | Once weekly | 4 weeks | Systemic |

TABLE 9
GLP-1 RA dose escalation regimen

| | First | Second | Third | Fourth | Fifth | Sixth | Seventh | Eighth |
|---|---|---|---|---|---|---|---|---|
| | dosage | dosage | dosage | dosage | dosage | dosage | dosage | dosage |
| GLP-1 RA | regimen | regimen | regimen | regimen | regimen | regimen | regimen | regimen |
| Exenatide | 5 μg twice | 10 μg | | | | | | |
| (IR) | daily; 4 | twice | | | | | | |
| | weeks | daily; 4 | | | | | | |
| | | weeks | | | | | | |
| Lixisenatide | 10 μg | 20 μg | | | | | | |
| | daily; 14 | daily; 14 | | | | | | |
| | days | days | | | | | | |
| Liraglutide | 600 μg | 1.2 mg | 1.8 mg | 2.4 mg | 3 mg | | | |
| | daily; 7 | daily; 7 | daily; 7 | daily; 7 | daily; 7 | | | |
| | days | days | days | days | days | | | |
| Semaglutide | 250 μg | 500 μg | 1 mg | 1.7 mg | 2.4 mg | 3 mg | 7 mg | 14 mg |
| | weekly; 4 | weekly; | weekly; | weekly; | weekly; | weekly; | weekly; | weekly; |
| | weeks | 4 weeks | 4 weeks | 4 | 4 | 4 | 4 | 4 |
| | | | | weeks | weeks | weeks | weeks | weeks |
| Dulaglutide | 750 μg | 1.5 mg | 3 mg | 4.5 mg | | | | |
| | weekly; 4 | weekly; | weekly; | weekly; | | | | |
| | weeks | 4 weeks | 4 weeks | 4 | | | | |
| | | | | weeks | | | | |
| Tirzepatide | 4 mg | 8 mg | 12 mg | | | | | |
| Dose | weekly; 4 | weekly; | weekly; | | | | | |
| Escalation | weeks | 4 weeks | 4 weeks | | | | | |
| Regimen A | | | | | | | | |
| Tirzepatide | 2.5 mg | 5 mg | 10 mg | 15 mg | | | | |
| Dose | weekly; 4 | weekly; | weekly; | weekly; | | | | |
| Escalation | weeks | 4 weeks | 4 weeks | 4 | | | | |
| Regimen B | | | | weeks | | | | |

In some embodiments, the GLP-1 RA is administered at a dose lower than the recommended dose shown in Tables 1-9. In some embodiments, the GLP-1 RA is administered at a dosage of from about 10% to about 90% (e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 81%, about 82%, about 83%, about 84%, about 85%, about 86%, about 87%, about 88%, about 89%, or about 90%) of the dose shown in Tables 1-9).

In some embodiments, the GLP-1 RA is exenatide formulated for immediate release and the treatment regimen with exenatide formulated for immediate release includes administering systemically a dose of n*5 μg wherein n has a value of from 1 to 5 (e.g., 1, 2, 3, 4, or 5). In some embodiments, the GLP-1 RA is exenatide formulated for extended release and the treatment regimen with exenatide formulated for extended release includes administering systemically a dose of n*2 mg wherein n has a value of 1.

In some embodiments, the GLP-1 RA is lixisenatide and the treatment regimen with lixisenatide includes administering systemically a dose of n*10 μg wherein n has a value of from 1 to 5 (e.g., 1, 2, 3, 4, or 5).

In some embodiments, the GLP-1 RA is liraglutide and the treatment regimen with liraglutide includes administering systemically a dose of n*600 μg wherein n has a value of from 1 to 10 (e.g., 1, 2, 3, 4, 5, 6, 7, 8, 9, or 10).

In some embodiments, the GLP-1 RA is semaglutide and the treatment regimen with semaglutide includes administering systemically a dose of n*250 μg wherein n has a value of from 1 to 100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 66, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100).

In some embodiments, the GLP-1 RA is dulaglutide and the treatment regimen with dulaglutide includes administering systemically a dose of n*750 μg wherein n has a value of from 1 to 10 (e.g., 1, 2, 3, 4, 5, 6, 7, 8, 9, or 10).

In some embodiments, the GLP-1 RA is tirzepatide and it is administered using one of two separate dose escalation regimens A and B. Treatment regimen A with tirzepatide includes administering systemically a dose of n*4 mg wherein n has a value of from 1 to 10 (e.g., 1, 2, 3, 4, 5, 6, 7, 8, 9, or 10). Treatment regimen B with tirzepatide includes administering systemically a dose of n*2.5 mg wherein n has a value of from 1 to 10 (e.g., 1, 2, 3, 4, 5, 6, 7, 8, 9, or 10).

In some embodiments, the GLP-1 RA is exenatide formulated for immediate release and the treatment regimen with exenatide formulated for immediate release includes administering systemically a dose of n*5 μg wherein n has a value of 1 for the first dose regimen and a value of 2 for the second dose regimen. In some embodiments, the GLP-1 RA is exenatide formulated for extended release and the treatment regimen with exenatide formulated for extended release includes administering systemically a dose of n*2 mg wherein n has a value of 1 for the first dose regimen.

In some embodiments, the GLP-1 RA is lixisenatide and the treatment regimen with lixisenatide includes administering systemically a dose of n*10 μg wherein n has a value of 1 for the first dose regimen and a value of 2 for the second dose regimen.

In some embodiments, the GLP-1 RA is liraglutide and the treatment regimen with liraglutide includes administering systemically a dose of n*600 μg wherein n has a value of 1 for the first dose regimen, a value of 2 for the second dose regimen, a value of 3 for the third dose regimen, a value of 4 for the fourth dose regimen, and a value of 5 for the fifth dose regimen.

In some embodiments, the GLP-1 RA is semaglutide and the treatment regimen with semaglutide includes administering systemically a dose of n*250 μg wherein n has a value of 1 for the first dose regimen, a value of 2 for the second dose regimen, a value of 4 for the third dose regimen, a value of 7 for the fourth dose regimen, a value of 10 for the fifth dose regimen, a value of 12 for the sixth dose regimen, a value of 28 for the seventh dose regimen, and a value of 56 for the eighth dose regimen.

In some embodiments, the GLP-1 RA is dulaglutide and the treatment regimen with dulaglutide includes administering systemically a dose of n*750 μg wherein n has a value of 1 for the first dose regimen, a value of 2 for the second dose regimen, a value of 4 for the third dose regimen, and a value of 6 for the fourth dose regimen.

In some embodiments, the GLP-1 RA is tirzepatide and treatment regimen A with tirzepatide includes administering systemically a dose of n*4 mg wherein n has a value of 1 for the first dose regimen, a value of 2 for the second dose regimen, and a value of 3 for the third dose regimen, or treatment regimen B with tirzepatide includes administering systemically a dose of n*2.5 mg wherein n has a value of 1 for the first dose regimen, a value of 2 for the second dose regimen, a value of 4 for the third dose regimen, and a value of 6 for the fourth dose regimen.

### Dosing and Routes of Administration

The method of inducing satiety and treating a condition, such as metabolic syndrome, diabetes, obesity, and obesity-related disorders includes using a combination therapy of any one of the systemically administered GLP-1 RAs at a dosing regimen as shown in Table 9 and topical-lingual administration of PYY in a dose of from about 2.5 μg to about 2.5 mg. In some embodiments, the combination therapy herein described includes the topical-lingual administration of PYY in a dose of from about 10 μg to about 1 mg. In some embodiments, the combination therapy herein described includes the topical-lingual administration of PYY in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of PYY(3-36) in a dose of from about 2.5 μg to about 2.5 mg. In some embodiments, the combination therapy herein described includes the topical-lingual administration of PYY(3-36) in a dose of from about 10 μg to about 1 mg. In some embodiments, the combination therapy herein described includes the topical-lingual administration of PYY(3-36) in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of PYY(3-36) in a dose of from about 2.5 μg to about 2.5 mg. In some embodiments, the combination therapy herein described includes the topical-lingual administration of PYY(3-36) in a dose of from about 10 μg to about 1 mg. In some embodiments, the combination therapy herein described includes the topical-lingual administration of PYY(3-36) in a dose of from about 25 μg to about 250 μg.

In some embodiments, the methods herein described a combination therapy of any one of the systemically administered GLP-1 RAs at a dosing regimen as shown in Table 9 and topical-lingual administration of leptin in a dose of from about 2.5 μg to about 2.5 mg. In some embodiments, the combination therapy herein described includes the topical-lingual administration of leptin in a dose of from about 10 μg to about 1 mg. In some embodiments, the combination therapy herein described includes the topical-lingual administration of leptin in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of OXM in a dose of from about 2.5 μg to about 2.5 mg. In some embodiments, the combination therapy herein described includes the topical-lingual administration of OXM in a dose of from about 10 μg to about 1 mg. In some embodiments, the combination therapy herein described includes the topical-lingual administration of OXM in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of CCK in a dose of from about 2.5 μg to about 2.5 mg. In some embodiments, the combination therapy herein described includes the topical-lingual administration of CCK in a dose of from about 10 μg to about 1 mg. In some embodiments, the combination therapy herein described includes the topical-lingual administration of CCK in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of insulin in a dose of from about 2.5 μg to about 2.5 mg. In some embodiments, the combination therapy herein described includes the topical-lingual administration of insulin in a dose of from about 10 μg to about 1 mg. In some embodiments, the combination therapy herein described includes the topical-lingual administration of insulin in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of amylin in a dose of from about 2.5 μg to about 2.5 mg. In some embodiments, the combination therapy herein described includes the topical-lingual administration of amylin in a dose of from about 10 μg to about 1 mg. In some embodiments, the combination therapy herein described includes the topical-lingual administration of amylin in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of GIP in a dose of from about 2.5 μg to about 2.5 mg. In some embodiments, the combination therapy herein described includes the topical-lingual administration of GIP in a dose of from about 10 μg to about 1 mg. In some embodiments, the combination therapy herein described includes the topical-lingual administration of GIP in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

In some embodiments, the methods herein described a combination therapy of any one of the systemically administered GLP-1 RAs at a dosing regimen as shown in Table 9 and topical-lingual administration of glucagon in a dose of from about 2.5 μg to about 2.5 mg. In some embodiments, the combination therapy herein described includes the topical-lingual administration of glucagon in a dose of from about 10 μg to about 1 mg. In some embodiments, the combination therapy herein described includes the topical-lingual administration of glucagon in a dose of from about 25 μg to about 250 μg (e.g., a dose of about 25 μg, 50 μg, 75 μg, 100 μg, 125 μg, 150 μg, 175 μg, 200 μg, 225 μg, or 250 μg).

The metabolic hormones disclosed herein are administered to the subject in combination with a GLP-1 RA. The metabolic hormone can be administered at the same time (e.g., administration of all active ingredients of the compositions herein described occurs within 15 minutes, 10 minutes, 5 minutes, 2 minutes, 1 minute, 30 seconds, 20 seconds, 10 seconds, 5 seconds, 2 seconds or less) of the GLP-1-RA. The GLP-1 RA and the metabolic hormone can also be administered sequentially, such that the action of the two overlaps and their combined effect is such that the reduction in a symptom, or other parameter related to the disorder is greater than what would be observed with one agent or treatment delivered alone or in the absence of the other. The GLP-1 RA and the metabolic hormone can be administered by different routes. For example, a composition of GLP-1 RA may be administered by intravenous injection while the metabolic hormone can be locally and topically administered to the tongue. The GLP-1 RA may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the metabolic hormone. In some embodiments, the metabolic hormone is administered after the GLP-1 RA. In some embodiments, the metabolic hormone is administered before the GLP-1 RA. In some embodiments, the metabolic hormone is administered at the same time as the GLP-1 RA.

The method herein described includes administration of a metabolic hormone (e.g., PYY, PYY(3-36), leptin, OXM, CCK, insulin, amylin, GIP, glucagon, an analog, variant, or biologically active fragment thereof locally to the mouth of a subject (e.g., tongue, salivary glands, lingual and/or sublingual epithelium, or mucosa) wherein the local administration does not produce substantial change to the level of the metabolic hormone in the blood and/or plasma of the subject. In general, the metabolic hormone level in the blood and/or plasma of the subject does not increase more than up to 10% (e.g., 9%, 8%, 7%, 6%, 5%, 4%, 3%, 2%, 1%, or less) of the pre-administration level of metabolic hormone. In some embodiments, the blood and/or plasma level of PYY(3-36) does not substantially surpass pre-prandial levels of from about 15 pmol/l to about 25 pmol/l as reported in Batterham et al, Cell Metabolism, 4:223-233, 2006, herein incorporated by reference, in its entirety, after topical-lingual administration of PYY(3-36). In some embodiments, the blood and/or plasma level of leptin does not substantially surpass pre-prandial levels of from about 5 ng/ml to about 35 ng/ml as reported in Considine et al, N. Engl. J. Med. 334:292-295, 1996, herein incorporated by reference in its entirety, after topical-lingual administration of leptin. In some embodiments, the blood and/or plasma level of OXM does not substantially surpass pre-prandial levels of from about 13.5 pmol/l to about 30.8 pmol/l as reported in Le Quellec et al, J. Clin. Endocrinol. Metab. 74:1405-1409, 1992, herein incorporated by reference in its entirety, after topical-lingual administration of OXM. In some embodiments, the blood and/or plasma level of CCK does not substantially surpass pre-prandial levels of about 2 μmol/ml as reported in Cuntz, U., et al, PLOS ONE. 8: e54457, 2013, herein incorporated by reference in its entirety, after topical-lingual administration of CCK. In some embodiments, the blood and/or plasma level of insulin does not substantially surpass pre-prandial levels of from about 2 mlU/ml to about 20 mlU/ml, as reported in Cryer, P. E., Williams Textbook of Endocrinology, 13^{th} Edition, pg. 1582-1607, 2016, herein incorporated by reference in its entirety, after topical-lingual administration of insulin. In some embodiments, the blood and/or plasma level of amylin does not substantially surpass pre-prandial levels of about 20 pmol/l as reported in Cooper et al, Hypertension, 26:460-464, 1995, herein incorporated by reference in its entirety, after topical-lingual administration of amylin. In some embodiments, the blood and/or plasma level of GIP does not substantially surpass pre-prandial levels of about 33 pmol/l as reported in Theodorakis et al, Diabetes Care, 27:1692-1698, 2004, herein incorporated by reference in its entirety, after topical-lingual administration of GIP. In some embodiments, the blood and/or plasma level of glucagon does not substantially surpass pre-prandial levels of about 33 pmol/l as reported in Jensen et al, Encyclopedia of Endocrine Diseases, Second Edition, Vol. 1:597-616, 2018, herein incorporated by reference in its entirety, after topical-lingual administration of glucagon.

In some embodiments, the method herein described includes administering the combination therapy before a meal. The feeling of fullness can last at least about 30, 60, 90, or 120 minutes or longer after administration with a GLP-1RA and a metabolic hormone and after the subject has eaten a meal. In some embodiments, the combination therapy is administered after a meal. In some embodiments, the combination therapy is administered at a regular schedule and independent of the subject's meal time.

The combination therapy of a subject using a locally administered metabolic hormone and a systemically administered GLP-1 RA reduce side effects associated with systemic GLP-1 RA monotherapy. In some embodiments, the combination therapy prevents, delays the onset, reduces the severity, and/or eliminates systemic GLP-1 monotherapy side effects such as one or more from the list composed of nausea, vomiting, diarrhea, abdominal pain, constipation, pancreatitis, diabetic retinopathy complications, hypoglycemia, acute kidney injury, or hypersensitivity.

### Kits

The methods herein described can be provided in a kit for use in satiety induction and/or treating a condition selected from metabolic syndrome, diabetes, obesity, and an obesity-related disorder. The kit may include one or more doses of the combination therapies as described herein. The kit may include one or more compositions including a metabolic hormone formulated for topical administration to a tongue (e.g., a ZYDIS® ODT) in a dose of from about 2.5 μg to about 2.5 mg (e.g., from about 10 μg to about 1 mg, e.g., from about 25 μg to about 250 μg, e.g., about 25 μg, 50 μg, 75 μg, 100 μg, 125 μg, 150 μg, 175 μg, 200 μg, 225 μg, or 250 μg), and a dose according to a first dose regimen, a second dose regimen, a third dose regimen, a fourth dose regimen, a fifth dose regimen, a sixth dose regimen, a seventh dose regimen, or an eighth dose regimen of a GLP-1 RA formulated for systemic administration, e.g., an injection pen (see Tables 1-9). The kit may include one or more compositions including a metabolic hormone formulated for topical administration to a tongue (e.g., a ZYDIS® ODT) at a dose of from about 10 μg to about 1 mg, and a dose according to a first dose regimen, a second dose regimen, a third dose regimen, a fourth dose regimen, a fifth dose regimen, a sixth dose regimen, a seventh dose regimen, or an eighth dose regimen of a GLP-1 RA formulated for systemic administration, e.g., an injection pen (see Tables 1-9). The kit may include one or more compositions including a metabolic hormone formulated for topical administration to a tongue (e.g., a ZYDIS® ODT) in a dose of from about 25 μg to about 250 μg (e.g., a dose of about 25 μg, 50 μg, 75 μg, 100 μg, 125 μg, 150 μg, 175 μg, 200 μg, 225 μg, or 250 μg), and a dose according to a first dose regimen, a second dose regimen, a third dose regimen, a fourth dose regimen, a fifth dose regimen, a sixth dose regimen, a seventh dose regimen, or an eighth dose regimen of a GLP-1 RA formulated for systemic administration, e.g., an injection pen (see Tables 1-9). The kit can include a package insert that instructs a user of the kit, such as a physician, to perform any one of the methods described herein. The kit may optionally include a syringe or other device for administering the composition. In some embodiments, the kit may include one or more additional therapeutic agents.

### EXAMPLES

The following examples are put forth so as to provide those of ordinary skill in the art with a description of how the methods described herein may be used, made, and evaluated, and are intended to be purely exemplary of the invention and are not intended to limit the scope of what the inventors regard as their invention.

### Example 1. Combination Therapy of a Patient with Type 2 Diabetes

A person recently diagnosed with type 2 diabetes mellitus completes a dose escalation regimen of liraglutide (VICTOZA®). The person's glycemic control reaches a steady state level and his endocrinologist recommends a combination therapy to improve the patient's condition. The person takes 3 mg of liraglutide (VICTOZA®) daily via an injection pen that delivers the GLP-1 RA systemically. Additionally, the person begins to take one rapidly dissolvable tablet with PYY(3-36) as the active ingredient at a dose of 25 μg. The tablet dissolves on the tongue of the person within 10 seconds. The person takes both liraglutide (VICTOZA®) and PYY(3-36) at the same time, such as within 5 minutes of each other, and once a day. The endocrinologist measures the person's glycemic control after 7 days of the combination therapy and observes a more than additive effect on the person's condition.

### Example 2. Combination Therapy of a Patient with Type 2 Diabetes and Obesity

A diabetic person recently diagnosed with obesity is at an increased risk of developing adverse cardiovascular conditions (e.g., heart attack or stroke) and starts a dose escalation regimen of dulaglutide (TRULICITY®). The person reaches a steady state level after receiving the second dose regimen of TRULICITY® but has suffered from various side effects due to the systemic administration of the GLP-1 RA. Her endocrinologist recommends a combination therapy to improve the patient's condition without raising the dose of TRULICITY®. The person is currently taking 1.5 mg of TRULICITY® weekly via an injection pen that delivers the GLP-1 RA systemically. Additionally, the person begins to take one rapidly dissolvable tablet with PYY(3-36) as the active ingredient at a dose of 200 μg. The tablet dissolves on the tongue of the person within 20 seconds. The person takes both TRULICITY® and PYY(3-36) at the same time, usually within 5 minutes of each other, and once a week. The endocrinologist measures the person's blood glucose levels after 4 weeks of the combination therapy and observes a more than additive effect on the person's condition.

SEQUENCES

Exenatide
SEQ ID NO: 1
HGEGTFTSDLSKQMEEEAVRLFIEWLKNGGPSSGAPPPS
Lixisenatide
SEQ ID NO: 2
HGEGTFTSDLSKQMEEEAVRLFIEWLKNGGPSSGAPPSKKKKKK
Liraglutide
SEQ ID NO: 3
HAEGTFTSDVSSYLEGQAAKEFIAWLVRGRG
K20 is modified with a glutamate palmitic acid side chain
Semaglutide
SEQ ID NO: 4
HXEGTFTSDVSSYLEGQAAKEFIAWLVRGRG
X = α-aminoisobutyric acid and K20 is acylated with a stearic diacid
Dulaglutide
SEQ ID NO: 5
HGEGTFTSDVSSYLEEQAAKEFIAWLVKGGGGGGGSGGGGSGGGGSAESKYGPPCPPCPAPEAAGGP
SVFLFPPKPKDTLMISRTPEVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKSLSLSL
G
Tirzepatide
SEQ ID NO: 6
YXEGTFTSDYSIXLDKIAQKAFVQWLIAGGPSSGAPPPS
X = α-aminoisobutyric acid at positions 2 and 13 and K20 contains a C20 fatty diacid conjugated via a
glutamate linker
PYY
SEQ ID NO: 7
MVFVRRPWPALTTVLLALLVCLGALVDAYPIKPEAPREDASPEELNRYYASLRHYLNLVTRQRYGKRDGP
DTLLSKTFFPDGEDRPVRSRSEGPDLW
[Pro34]PYY
SEQ ID NO: 8
YPIKPEAPGEDASPEELNRYYASLRHYLNLVTRPRY
PYY(3-36)
SEQ ID NO: 9
IKPEAPGEDASPEELNRYYASLRHYLNLVTRQRY
Leptin
SEQ ID NO: 10
MHWGTLCGFLWLWPYLFYVQAVPIQKVQDDTKTLIKTIVTRINDISHTQSVSSKQKVTGLDFIPGLHPILTL
SKMDQTLAVYQQILTSMPSRNVIQISNDLENLRDLLHVLAFSKSCHLPWASGLETLDSLGGVLEASGYSTE
VVALSRLQGSLQDMLWQLDLSPGC
OXM
SEQ ID NO: 11
HSQGTFTSDYSKYLDSRRAQDFVQWLMNTKRNKNNIA
CCK
SEQ ID NO: 12
MNSGVCLCVLMAVLAAGALTQPVPPADPAGSGLQRAEEAPRRQLRVSQRTDGESRAHLGALLARYIQQ
ARKAPSGRMSIVKNLQNLDPSHRISDRDYMGWMDFGRRSAEEYEYPS
Insulin
SEQ ID NO: 13
MALWMRLLPLLALLALWGPDPAAAFVNQHLCGSHLVEALYLVCGERGFFYTPKTRREAEDLQVGQVELG
GGPGAGSLQPLALEGSLQKRGIVEQCCTSICSLYQLENYCN
Amylin
SEQ ID NO: 14
MGILKLQVFLIVLSVALNHLKATPIESHQVEKRKCNTATCATQRLANFLVHSSNNFGAILSSTNVGSNTYGK
RNAVEVLKREPLNYLPL
GIP
SEQ ID NO: 15
MTTSPILQLLLRLSLCGLLLQRAETGSKGQTAGELYQRWERYRRECQETLAAAEPPSGLACNGSFDMYV
CWDYAAPNATARASCPWYLPWHHHVAAGFVLRQCGSDGQWGLWRDHTQCENPEKNEAFLDQRLILER
LQVMYTVGYSLSLATLLLALLILSLFRRLHCTRNYIHINLFTSFMLRAAAILSRDRLLPRPGPYLGDQALALW
NQALAACRTAQIVTQYCVGANYTWLLVEGVYLHSLLVLVGGSEEGHFRYYLLLGWGAPALFVIPWVIVRY
LYENTQCWERNEVKAIWWIIRTPILMTILINFLIFIRILGILLSKLRTRQMRCRDYRLRLARSTLTLVPLLGVHE
VVFAPVTEEQARGALRFAKLGFEIFLSSFQGFLVSVLYCFINKEVQSEIRRGWHHCRLRRSLGEEQRQLP
ERAFRALPSGSGPGEVPTSRGLSSGTLPGPGNEASRELESYC
Glucagon
SEQ ID NO: 16
MKSIYFVAGLFVMLVQGSWQRSLQDTEEKSRSFSASQADPLSDPDQMNEDKRHSQGTFTSDYSKYLDS
RRAQDFVQWLMNTKRNRNNIAKRHDEFERHAEGTFTSDVSSYLEGQAAKEFIAWLVKGRGRRDFPEEV
AIVEELGRRHADGSFSDEMNTILDNLAARDFINWLIQTKITDRK
GLP-1
SEQ ID NO: 17
MKSIYFVAGLFVMLVQGSWQRSLQDTEEKSRSFSASQADPLSDPDQMNED
KRHSQGTFTSDYSKYLDSRRAQDFVQWLMNTKRNRNNIAKRHDEFERHAE
GTFTSDVSSYLEGQAAKEFIAWLVKGRGRRDFPEEVAIVEELGRRHADGS
FSDEMNTILDNLAARDFINWLIQTKITDRK
Metreleptin
SEQ ID NO: 18
MVPIQKVQDDTKTLIKTIVTRINDISHTQSVSSKQKVTGLDFIPGLHPILTLSKMDQTLA
VYQQILTSMPSRNVIQISNDLENLRDLLHVLAFSKSCHLPWASGLETLDSLGGVLEASGY
STEVVALSRLQGSLQDMLWQLDLSPGC
Insulin Aspart
A chain
SEQ ID NO: 19
GIVEQCCTSICSLYQLENYCN
Insulin Aspart
B chain
SEQ ID NO: 20
FVNQHLCGSHLVEALYLVCGERGFFYTDKT
Insulin Glargine
A chain
SEQ ID NO: 21
GIVEQCCTSICSLYQLENYCG
Insulin Glargine
B chain
SEQ ID NO: 22
FVNQHLCGSHLVEALYLVCGERGFFYTPKTRR
Insulin Lispro
A chain
SEQ ID NO: 23
GIVEQCCTSICSLYQLENYCN
Insulin Lispro
B chain
SEQ ID NO: 24
FVNQHLCGSHLVEALYLVCGERGFFYTKPT
Insulin Glulisine
A chain
SEQ ID NO: 25
GIVEQCCTSICSLYQLENYCN
Insulin Glulisine
B chain
SEQ ID NO: 26
FVKQHLCGSHLVEALYLVCGERGFFYTPET
Insulin Detemir
A chain
SEQ ID NO: 27
GIVEQCCTSICSLYQLENYCN
Insulin Detemir
B chain
SEQ ID NO: 28
FVNQHLCGSHLVEALYLVCGERGFFYTPK
Pramlintide
SEQ ID NO: 29
KCNTATCATQRLANFLVHSSNNFGPILPPTNVGSNTY
Dasiglucagon
SEQ ID NO: 30
HSQGTFTSDYSKYLDXARAEEFVKWLEST
X = α-aminoisobutyric acid

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A method of inducing satiety or treating a condition selected from metabolic syndrome, diabetes, obesity, an obesity-related disorder, nonalcoholic steatohepatitis (NASH), chronic kidney disease (CKD), polycystic ovary syndrome (PCOS), cardiovascular disease (CVD), obstructive sleep apnea (OSA), retinopathy, peripheral vascular disease (PVD), peripheral artery disease (PAD), and neuropathy, the method comprising topical-lingual administration of a dose of from 2.5 μg to 250 μg of a metabolic hormone to a subject under a glucagon-like peptide 1 receptor agonist (GLP-1 RA) treatment regimen of any one of Tables 1, 2, 3, 4, 5, 6, 7 or 8.

2. The method of claim 1, comprising topical-lingual administration of a dose of from 2.5 μg to 250 μg of a metabolic hormone to a subject under a GLP-1 RA treatment regimen of Table 2.

3. The method of any one of claims 1-2, comprising topical-lingual administration of a dose of from 2.5 μg to 250 μg of a metabolic hormone to a subject under a GLP-1 RA treatment regimen of Table 3.

4. The method of any one of claims 1-3, comprising topical-lingual administration of a dose of from 2.5 μg to 250 μg of a metabolic hormone to a subject under a GLP-1 RA treatment regimen of Table 4.

5. The method of any one of claims 1-4, comprising topical-lingual administration of a dose of from 2.5 μg to 250 μg of a metabolic hormone to a subject under a GLP-1 RA treatment regimen of Table 5.

6. The method of any one of claims 1-5, comprising topical-lingual administration of a dose of from 2.5 μg to 250 μg of a metabolic hormone to a subject under a GLP-1 RA treatment regimen of Table 6.

7. The method of any one of claims 1-6, comprising topical-lingual administration of a dose of from 2.5 μg to 250 μg of a metabolic hormone to a subject under a GLP-1 RA treatment regimen of Table 7.

8. The method of any one of claims 1-7, comprising topical-lingual administration of a dose of from 2.5 μg to 250 μg of a metabolic hormone to a subject under a GLP-1 RA treatment regimen of Table 8.

9. The method of any one of claims 1-8, wherein the metabolic hormone targets a Y2 receptor of the subject.

10. The method of claim 9, wherein the Y2 receptor is associated with a tongue of the subject.

11. The method of any one of claims 1-10, wherein the topical-lingual administration of the metabolic hormone produces substantially no change in the level of metabolic hormone in the blood and/or plasma of the subject.

12. The method of any one of claims 1-11, wherein the metabolic hormone is PYY, PYY(3-36), leptin, oxyntomodulin (OXM), cholecystokinin (CCK), insulin, amylin, gastric inhibitory peptide (GIP), glucagon, an analog, variant, or biologically active fragment thereof.

13. The method of any one of claims 1-12, wherein 2.5 μg, 10 μg, 25 μg, 50 μg, 100 μg, or 250 μg of the metabolic hormone is administered.

14. The method of any one of claims 1-13, wherein the metabolic hormone is formulated as an orally dissolvable tablet (ODT), a lozenge, a film, or a spray.

15. The method of claim 14, wherein the ODT is in a rapidly dissolving form comprising partially hydrolyzed gelatin at a concentration of from about 1% to 6% w/v, mannitol, hydrolyzed dextran, alginate, polyvinyl alcohol, polyvinylpyrrolidone, acacia, aspartame, sodium methylparaben, sodium propylparaben, phenylalanine, water, or a combination thereof.

16. The method of any one of claims 1-15, wherein the method reduces a GLP-1-associated side effect.

17. The method of claim 16, wherein the GLP-1-associated side effect comprises nausea, vomiting, diarrhea, abdominal pain, constipation, pancreatitis, diabetic retinopathy complications, hypoglycemia, acute kidney injury, hypersensitivity, or a combination thereof.

18. The method of any one of claims 1-17, wherein the GLP-1 receptor agonist is formulated as a tablet, a gel capsule, or a liquid.

19. A method of inducing satiety or treating a condition selected from metabolic syndrome, diabetes, obesity, an obesity-related disorder, NASH, CKD, PCOS, CVD, OSA, retinopathy, PVD, PAD, and neuropathy comprising topical-lingual administration of a dose of from 2.5 μg to 250 μg of a metabolic hormone to a subject that has completed a systemic GLP-1 RA dose escalation regimen of Table 9.

20. A method of inducing satiety or treating a condition selected from metabolic syndrome, diabetes, obesity, an obesity-related disorder, NASH, CKD, PCOS, CVD, OSA, retinopathy, PVD, PAD, and neuropathy comprising topical-lingual administration of a dose of from 2.5 μg to 250 μg of a metabolic hormone to a subject under a GLP-1 RA treatment regimen wherein:
(a) the GLP-1 RA is exenatide formulated for immediate release, and the treatment regimen comprises systemically administering n*5 μg of exenatide, wherein n has a value of from 1-2;
(b) the GLP-1 RA is exenatide formulated for extended release, and the treatment regimen comprises systemically administering n*2 mg of exenatide, wherein n has a value of 1;
(c) the GLP-1 RA is lixisenatide and the treatment regimen comprises systemically administering n*10 μg of lixisenatide, wherein n has a value of from 1-2;
(d) the GLP-1 RA is liraglutide and the treatment regimen comprises systemically administering n*600 μg of liraglutide, wherein n has a value of from 1-10;
(e) the GLP-1 RA is semaglutide and the treatment regimen comprises systemically administering n*250 μg of semaglutide, wherein n has a value of from 1-100;
(f) the GLP-1 RA is dulaglutide and the treatment regimen comprises systemically administering n*750 μg of dulaglutide, wherein n has a value of from 1-10;
(g) the GLP-1 RA is tirzepatide and treatment regimen A comprises systemically administering n*4 mg, n*8 mg, or n*12 mg, and treatment regimen B comprises systemically administering n*2.5 mg, n*5 mg, n*10 mg, or n*15 mg of tirzepatide, wherein n has a value of from 1-10.

21. The method of claim 20, wherein:
(a) the GLP-1 RA is semaglutide and the treatment regimen comprises systemically administering n*1.7 μg of semaglutide, wherein n has a value of from 1-10; or
(b) the GLP-1 RA is semaglutide and the treatment regimen comprises systemically administering n*2.4 μg of semaglutide, wherein n has a value of from 1-10.

22. A method of inducing satiety or treating a condition selected from metabolic syndrome, diabetes, obesity, an obesity-related disorder, NASH, CKD, PCOS, CVD, OSA, retinopathy, PVD, PAD, and neuropathy in a subject comprising:
(a) systemic administration from 10% to 90% of a GLP-1 RA to the subject receiving a first dosage regimen of Table 9; and
(b) topical-lingual administration of a dose of from 2.5 μg to 250 μg of a metabolic hormone.

23. The method of claim 22, comprising:
(a) systemic administration from 10% to 90% of a GLP-1 RA to the subject receiving a second dosage regimen of Table 9; and
(b) topical-lingual administration of a dose of from 2.5 μg to 250 μg of a metabolic hormone.

24. The method of any one of claims 22-23, comprising:
(a) systemic administration from 10% to 90% of a GLP-1 RA to the subject receiving a third dosage regimen of Table 9; and
(b) topical-lingual administration of a dose of from 2.5 μg to 250 μg of a metabolic hormone.

25. The method of any one of claims 22-24, comprising:
(a) systemic administration from 10% to 90% of a GLP-1 RA to the subject receiving a fourth dosage regimen of Table 9; and
(b) topical-lingual administration of a dose of from 2.5 μg to 250 μg of a metabolic hormone.

26. The method of any one of claims 22-25, comprising:
(a) systemic administration from 10% to 90% of a GLP-1 RA to the subject receiving a fifth dosage regimen of Table 9; and
(b) topical-lingual administration of a dose of from 2.5 μg to 250 μg of a metabolic hormone.

27. The method of any one of claims 22-26, comprising:
(a) systemic administration from 10% to 90% of a GLP-1 RA to the subject receiving a sixth dosage regimen of Table 9; and
(b) topical-lingual administration of a dose of from 2.5 μg to 250 μg of a metabolic hormone.

28. The method of any one of claims 22-27, comprising:
(a) systemic administration from 10% to 90% of a GLP-1 RA to the subject receiving a seventh dosage regimen of Table 9; and
(b) topical-lingual administration of a dose of from 2.5 μg to 250 μg of a metabolic hormone.

29. The method of any one of claims 22-28, comprising:
(a) systemic administration from 10% to 90% of a GLP-1 RA to the subject receiving an eighth dosage regimen of Table 9; and
(b) topical-lingual administration of a dose of from 2.5 μg to 250 μg of a metabolic hormone.

30. A kit comprising a metabolic hormone formulated for topical-lingual administration in a dose of from 2.5 μg to 250 μg and a dose of a GLP-1 RA formulated for systemic administration.


SEQUENCE LISTING (30 sequences)

SEQ ID NO: 1 - protein, 39 aa, Artificial Sequence
  Synthetic Construct
HGEGTFTSDLSKQMEEEAVRLFIEWLKNGGPSSGAPPPS

SEQ ID NO: 2 - protein, 44 aa, Artificial Sequence
  Synthetic Construct
HGEGTFTSDLSKQMEEEAVRLFIEWLKNGGPSSGAPPSKKKKKK

SEQ ID NO: 3 - protein, 31 aa, Artificial Sequence
  Synthetic Construct
HAEGTFTSDVSSYLEGQAAKEFIAWLVRGRG

SEQ ID NO: 4 - protein, 31 aa, Artificial Sequence
  Synthetic Construct
  MISC_FEATURE (2)..(2): Xaa is alpha-aminoisobutyric acid
HXEGTFTSDVSSYLEGQAAKEFIAWLVRGRG

SEQ ID NO: 5 - protein, 275 aa, Artificial Sequence
  Synthetic Construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 6 - protein, 39 aa, Artificial Sequence
  Synthetic Construct
  MISC_FEATURE (2)..(2): Xaa is alpha-aminoisobutyric acid
  MISC_FEATURE (13)..(13): Xaa is alpha-aminoisobutyric acid
YXEGTFTSDYSIXLDKIAQKAFVQWLIAGGPSSGAPPPS

SEQ ID NO: 7 - protein, 97 aa, Homo sapiens
MVFVRRPWPALTTVLLALLVCLGALVDAYPIKPEAPREDASPEELNRYYASLRHYLNLVTRQRYGKRDGPDTLLSKTFFPDGEDRPVRSRSEGPDLW

SEQ ID NO: 8 - protein, 36 aa, Artificial Sequence
  Synthetic Construct
YPIKPEAPGEDASPEELNRYYASLRHYLNLVTRPRY

SEQ ID NO: 9 - protein, 34 aa, Homo sapiens
IKPEAPGEDASPEELNRYYASLRHYLNLVTRQRY

SEQ ID NO: 10 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 11 - protein, 37 aa, Homo sapiens
HSQGTFTSDYSKYLDSRRAQDFVQWLMNTKRNKNNIA

SEQ ID NO: 12 - protein, 115 aa, Homo sapiens
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 13 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 14 - protein, 89 aa, Homo sapiens
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 15 - protein, 466 aa, Homo sapiens
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 16 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 17 - protein, 180 aa, Homo sapiens; identical to SEQ ID NO: 16

SEQ ID NO: 18 - protein, 147 aa, Artificial Sequence
  Synthetic Construct
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 19 - protein, 21 aa, Artificial Sequence
  Synthetic Construct
GIVEQCCTSICSLYQLENYCN

SEQ ID NO: 20 - protein, 30 aa, Artificial Sequence
  Synthetic Construct
FVNQHLCGSHLVEALYLVCGERGFFYTDKT

SEQ ID NO: 21 - protein, 21 aa, Artificial Sequence
  Synthetic Construct
GIVEQCCTSICSLYQLENYCG

SEQ ID NO: 22 - protein, 32 aa, Artificial Sequence
  Synthetic Construct
FVNQHLCGSHLVEALYLVCGERGFFYTPKTRR

SEQ ID NO: 23 - protein, 21 aa, Artificial Sequence
  Synthetic Construct
GIVEQCCTSICSLYQLENYCN

SEQ ID NO: 24 - protein, 30 aa, Artificial Sequence
  Synthetic Construct
FVNQHLCGSHLVEALYLVCGERGFFYTKPT

SEQ ID NO: 25 - protein, 21 aa, Artificial Sequence
  Synthetic Construct
GIVEQCCTSICSLYQLENYCN

SEQ ID NO: 26 - protein, 30 aa, Artificial Sequence
  Synthetic Construct
FVKQHLCGSHLVEALYLVCGERGFFYTPET

SEQ ID NO: 27 - protein, 21 aa, Artificial Sequence
  Synthetic Construct
GIVEQCCTSICSLYQLENYCN

SEQ ID NO: 28 - protein, 29 aa, Artificial Sequence
  Synthetic Construct
FVNQHLCGSHLVEALYLVCGERGFFYTPK

SEQ ID NO: 29 - protein, 37 aa, Artificial Sequence
  Synthetic Construct
KCNTATCATQRLANFLVHSSNNFGPILPPTNVGSNTY

SEQ ID NO: 30 - protein, 29 aa, Artificial Sequence
  Synthetic Construct
  MISC_FEATURE (16)..(16): Xaa is alpha-aminoisobutyric acid
HSQGTFTSDYSKYLDXARAEEFVKWLEST